(19) United States
(12) Patent Application Publication
(10) Pub. No.: US 2026/0158141 A1
(43) Pub. Date: Jun. 11, 2026

(54) USE OF THP IN PREPARATION OF MEDICAMENT FOR ENHANCING ANTITUMOR EFFICACY OF Vy9V§2 T CELLS

(71) Applicant: Xinxiang Medical University, Xinxiang (CN)

(72) Inventors: Feng REN, Xinxiang (CN); Tian WEI, Xinxiang (CN); Bingjie LIU, Xinxiang (CN); Zishan YANG, Xinxiang (CN); Tiesuo ZHAO, Xinxiang (CN); Zhili CHU, Xinxiang (CN); Zhinan YIN, Guangzhou (CN); Hongru ZHANG, Tianjin (CN); Jianlei HAO, Guangzhou (CN); Chenchen LYU, Xinxiang (CN); Junchan GAO, Xinxiang (CN); Mengjuan LI, Xinxiang (CN); Yuanhua QIN, Xinxiang (CN)

(73) Assignee: Xinxiang Medical University, Xinxiang (CN)

(21) Appl. No.: 19/412,822

(22) Filed: Dec. 8, 2025

(30) Foreign Application Priority Data
Dec. 9, 2024 (CN) 202411795942.3

(51) Int. Cl. A61K 40/11; A61K 31/7048; A61K 47/44; A61P 35/00; C12N 5/0783

(52) U.S. Cl. CPC A61K 40/11; A61K 31/7048; A61K 47/44; A61P 35/00; C12N 5/0636; C12N 2501/06; C12N 2501/2302

(57) ABSTRACT

A use of THP (Pirarubicin) in preparation of a medicament for enhancing antitumor efficacy of Vγ9Vδ2 T cells is provided. It is discovered that a surface marker MIC A/B of tumor cells treated with THP is significantly enhanced, and THP-induced tumor cells enable Vγ9Vδ2 T cells to highly express NKG2D. An NKG2D/MIC A/B pathway is a classical pathway through which Vγ9Vδ2 T cells exert a killing effect. THP significantly enhances an ability of Vγ9Vδ2 T cells to kill hepatocellular carcinoma cells through regulating the NKG2D/MIC A/B pathway, and provides a new therapeutic solution for TACE (transcatheter arterial chemoembolization) surgical treatment of clinical cancer patients.

### CROSS REFERENCE TO THE RELATED APPLICATIONS

This application is based upon and claims priority to Chinese Patent Application No. 202411795942.3, filed on Dec. 9, 2024, the entire contents of which are incorporated herein by reference.

### TECHNICAL FIELD

The present invention relates to the field of biopharmaceutical technology, and in particular, to use of THP in preparation of a medicament for enhancing antitumor efficacy of Vγ9Vδ2 T cells.

### BACKGROUND

Transcatheter arterial chemoembolization (TACE) is a preferred therapeutic method for intermediate and advanced hepatocellular carcinoma (HCC). Chemotherapeutic agents such as Pirarubicin (THP) used during the TACE procedure can directly damage DNA and induce apoptosis. Ischemia and hypoxia caused by TACE affect DNA synthesis of tumor cells, thereby promoting necrosis of tumor cells, activating T lymphocyte response, and altering tumor immune microenvironment. However, many patients still have poor prognosis after the TACE procedure; therefore, other therapeutic methods combined with TACE surgical treatment of hepatocellular carcinoma are urgently needed.

Studies have found that allogeneic Vγ9Vδ2 T cells can improve the prognosis of some HCC patients, and a high proportion of Vγ9Vδ2 T cells in peripheral blood of patients before the TACE procedure indicates good prognosis. In a tumor microenvironment of HCC, γδT cells can recognize stress-induced molecules MIC A/B expressed by tumor cells through surface-expressed NKG2D receptors and exert antitumor effects. A Vγ9Vδ2 T cell subgroup among γδT cells has potential to serve as a means of cellular immunotherapy and plays an important role in treatment of HCC. However, antitumor effect of Vγ9Vδ2 T cells is currently weak and further research is urgently needed to enhance antitumor effect of Vγ9Vδ2 T cells and to exert a better function of improving prognosis of HCC patients.

### SUMMARY

To achieve the foregoing objective, the present invention provides the following technical solutions.

The present invention provides use of THP in preparation of a medicament for enhancing antitumor efficacy of Vγ9Vδ2 T cells.

Preferably, the tumor includes one or more of hepatocellular carcinoma, malignant lymphoma, breast cancer, ovarian cancer, uterine cancer, gastric cancer, and bladder cancer.

Preferably, an effector-to-target ratio of Vγ9Vδ2 T cells to tumor cells is 0.5-12:1.

Preferably, the THP is used in a solution form.

Preferably, the THP solution includes THP and iodized oil, and a mass-to-volume ratio of the THP to the iodized oil is 2 μg:1-100 μL.

The present invention provides an antitumor medicament, which includes separately packaged THP and Vγ9Vδ2 T cells.

The present invention has the technical effects and advantages as follows.

The present invention provides use of THP in preparation of a medicament for enhancing antitumor efficacy of Vγ9Vδ2 T cells, and further discovers that a surface marker MIC A/B of tumor cells treated with THP is significantly enhanced, and THP-induced tumor cells enable Vγ9Vδ2 T cells to highly express NKG2D. An NKG2D/MIC A/B pathway is a classical pathway through which Vγ9Vδ2 T cells exert a killing effect. THP significantly enhances an ability of Vγ9Vδ2 T cells to kill hepatocellular carcinoma cells through regulating the NKG2D/MIC A/B pathway, and provides a new therapeutic solution for TACE (transcatheter arterial chemoembolization) surgical treatment of clinical cancer patients.

### BRIEF DESCRIPTION OF THE DRAWINGS

FIG. 1 is a diagram showing a killing effect of Vγ9Vδ2 T cells on Huh7 tumor cells at different effector-to-target ratios detected by flow cytometry;

FIG. 2 is a diagram showing a killing effect of Vγ9Vδ2 T cells on HepG2 tumor cells at different effector-to-target ratios detected by flow cytometry;

FIG. 3 is a diagram showing an effect of THP enhancing killing of Huh7 hepatocellular carcinoma cells by Vγ9Vδ2 T cells;

FIG. 4 is a diagram showing an effect of THP enhancing killing of HepG2 hepatocellular carcinoma cells by Vγ9Vδ2 T cells;

FIG. 5 is a diagram showing an effect of THP combined with Vγ9Vδ2 T cells significantly inhibiting proliferation of Huh7 tumor cells;

FIG. 6 is a diagram showing an effect of THP combined with Vγ9Vδ2 T cells significantly inhibiting proliferation of HepG2 tumor cells;

FIG. 7 is a diagram showing an effect of THP enhancing expression of MICA and MICB of Huh7 tumor cells;

FIG. 8 is a diagram showing an effect of THP enhancing expression of MICA and MICB of HepG2 tumor cells;

FIG. 9 is a diagram showing an effect of THP-treated Huh7 tumor cells promoting high expression of NKG2D of Vγ9Vδ2 T cells;

FIG. 10 is a diagram showing an effect of THP-treated HepG2 tumor cells promoting high expression of NKG2D of Vγ9Vδ2 T cells;

FIG. 11 is a diagram showing body weight change of hepatocellular carcinoma-bearing mice during treatment; and

FIG. 12 is a diagram showing an effect of THP enhancing an antitumor immune response ability of Vγ9Vδ2 T cells in hepatocellular carcinoma-bearing mice during treatment.

### DETAILED DESCRIPTION OF THE EMBODIMENTS

The present invention provides use of THP in preparation of a medicament for enhancing antitumor efficacy of Vγ9Vδ2 T cells. THP significantly enhances an ability of Vγ9Vδ2 T cells to kill hepatocellular carcinoma cells through regulating the NKG2D/MIC A/B pathway.

In the present invention, the tumor preferably includes one or more of hepatocellular carcinoma, malignant lymphoma, breast cancer, ovarian cancer, uterine cancer, gastric cancer, and bladder cancer.

In the present invention, the effector-to-target ratio of Vγ9Vδ2 T cells to tumor cells is preferably 0.5-12:1, further preferably 1-12:1, and more preferably 10:1.

In the present invention, the THP is preferably used in a solution form; the THP solution preferably includes THP and iodized oil; a mass-to-volume ratio of the THP to the iodized oil is preferably 2 μg:1-100 μL, further preferably 2 μg:10-80 μL, and more preferably 2 μg:50 μL; and the THP solution is preferably used in injection.

The present invention provides an antitumor medicament, which preferably includes separately packaged THP and Vγ9Vδ2 T cells. The treatment of tumor cells by THP enhances surface markers MIC A/B of cancer cells, and THP-induced tumor cells enable Vγ9Vδ2 T cells to highly express NKG2D. The THP significantly enhances an ability of Vγ9Vδ2 T cells to kill hepatocellular carcinoma cells through regulating the NKG2D/MIC A/B pathway.

The technical solutions provided by the present invention will be described in detail below with reference to examples, which, however, should not be construed as limiting the scope of the present invention.

### Materials Source

Huh7 cells were purchased from Shanghai Zhongqiao Xinzhou Biological Technology Co., Ltd., Cat. No. ZQ 0025.

HepG2 cells were purchased from Servicebio, Cat. No. STCC10114P.

CFSE was purchased from Sigma, Cat. No. 21888.

THP was purchased from Selleck, Cat. No. S1393.

Ficoll was purchased from Cytiva, Cat. No. 17144003.

Zol was purchased from Sigma-Aldrich, Cat. No. SML0223.

Recombinant IL-2 (rIL-2) was purchased from Beijing Four Rings Bio-pharmaceutical Co., Ltd.

RPMI 1640 complete medium (containing 10% FBS) was purchased from Gibco, Cat. No. 6124034.

Anti-TCR Vγ9Vδ2 T flow cytometry antibody was purchased from BioLegend, Cat. No. 331407.

Anti-CD3 flow cytometry antibody was purchased from BioLegend, Cat. No. 300454.

PI was purchased from BioLegend, Cat. No. 421301.

Ki67 flow cytometry antibody was purchased from BioLegend, Cat. No. 350505.

Reverse transcription kit was purchased from Servicebio, Cat. No. G3337-100.

SYBR Green was purchased from Servicebio, Cat. No. G3326-15.

Lysis buffer (RIPA:PMSF:protease inhibitor:phosphatase inhibitor=100:1:1:1) was purchased from Beyotime.

C-NKG mice (male, 4 weeks old) were purchased from Cyagen Biosciences (Suzhou) Inc.

Exposure solution was purchased from Kermel, Cat. No. M0101.

### Example 1: Killing Effect of Vγ9Vδ2 T Cells on HCC (Huh7, HepG2) Tumor Cells

### 1) In Vitro Expansion of Vγ9Vδ2 T Cells

Peripheral blood was collected from healthy donors with a body mass index (BMI) of 18.5-24.99, and PBMCs were isolated by Ficoll density gradient centrifugation. Subsequently, Vγ9Vδ2 T cells were expanded and cultured: on day 0, every 3×10⁶ Vγ9Vδ2 T cells were added with 50 μM Zol and 100 IU/mL rIL-2 in RPMI 1640 complete medium (containing 10% FBS), and cultured in a 37° C., 5% CO₂, and saturated humidity incubator. On days 3, 5, 7, and 9, the medium containing 100 IU/mL rIL-2 was replaced. After 9 days of induction, immunofluorescence double staining was performed using anti-TCR Vγ9Vδ2 T and anti-CD3 flow cytometry antibodies, and the purity of anti-TCR Vγ9Vδ2 T cells was analyzed by flow cytometry. Vγ9Vδ2 T cells with a proportion of 80% or higher were used for subsequent experiments.

### 2) Detection of Killing Effect of Vγ9Vδ2 T Cells on HCC (Huh7, HepG2) Tumor Cells by Flow Cytometry

Vγ9Vδ2 T cells (effector cells, E) were co-cultured with HepG2 and Huh7 cells (target cells, T) at effector-to-target (E:T) ratios of 0:1 (Vγ9Vδ2 T cells 0, tumor cells 2×10⁵), 1:1 (Vγ9Vδ2 T cells 0.2×10⁶, tumor cells 2×10⁵), 5:1 (Vγ9Vδ2 T cells 1×10⁶, tumor cells 2×10⁵), and 10:1 (Vγ9Vδ2 T cells 2×10⁶, tumor cells 2×10⁵). The cells were placed in round-bottom flow cytometry tubes and cultured in 150 μL RPMI 1640 medium without serum, and incubated at 37° C. in the dark for 5-6 h.

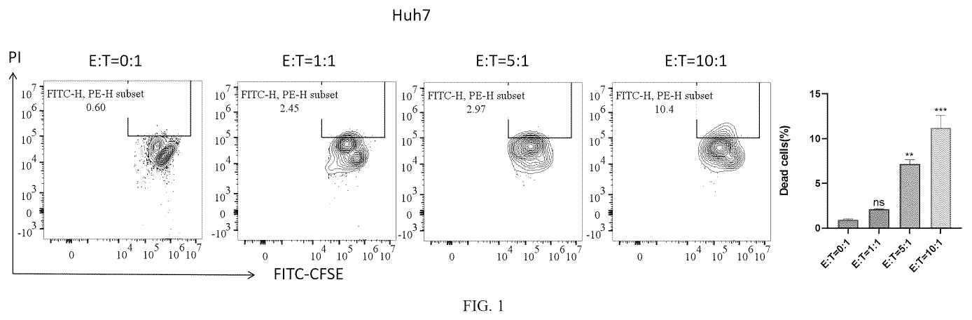

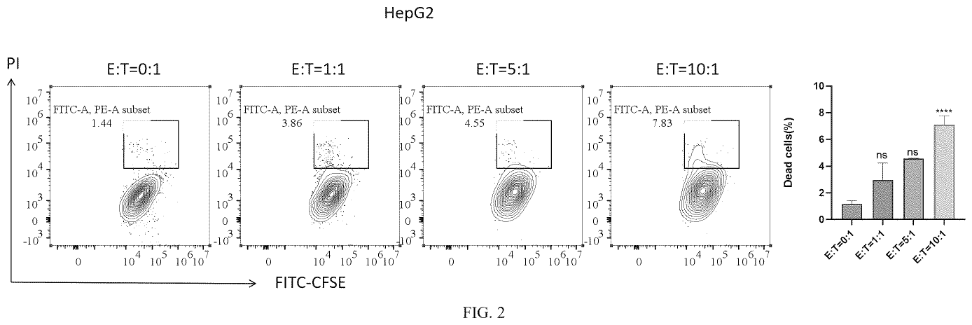

After co-culture, 1 mL PBS was added to resuspend, the cells were centrifuged, and the supernatant was discarded. Each tube was added with 100 μL PBS and 2 μL PI, stained in the dark at 4° C. for 10 min, then resuspended with 400 μL PBS, and analyzed by flow cytometry to detect the killing effect of Vγ9Vδ2 T cells on tumor cells (HepG2, Huh7) at different effector-to-target ratios. The results are shown in FIGS. 1 and 2.

FIGS. 1 and 2 show that the killing effect at the effector-to-target ratio of 10:1 is significantly enhanced compared with 0:1, 1:1, and 5:1.

### Example 2: Killing Effect of Vγ9Vδ2 T Cells on THP-Treated HCC (Huh7, HepG2) Tumor Cells and Effect on Tumor Cell Proliferation

### 1) Experiment Grouping

Blank control group: Tumor cells were plated at 3×10⁵ cells per well in a 12-well plate and incubated at 37° C., 5% CO₂ for 30 h.

THP group: Tumor cells were plated at 3×10⁵ cells per well in a 12-well plate and incubated at 37° C., 5% CO₂ for 18 h. After cell attachment, 2 μM THP was added to treat the cells for 12 h.

Vγ9Vδ2 T group: Tumor cells were plated at 3×10⁵ cells per well in a 12-well plate and incubated at 37° C., 5% CO₂ for 24 h. Then, 3×10⁵ tumor cells and 3×10⁶ Vγ9Vδ2 T cells were collected and co-cultured in round-bottom flow cytometry tubes at an effector-to-target ratio of 10:1. The culture volume was 150 μL. The cells were incubated at 37° C. in the dark for 6 h.

THP+Vγ9Vδ2 T group: Tumor cells were plated at 3×10⁵ cells per well in a 12-well plate and incubated at 37° C., 5% CO₂ for 12 h. After cell attachment, 2 μM THP was added to treat the cells for 12 h. Then, 3×10⁵ tumor cells and 3×10⁶ Vγ9Vδ2 T cells were collected and co-cultured with THP-treated HCC (HepG2 and Huh7) cells in round-bottom flow cytometry tubes at an effector-to-target ratio of 10:1. The culture volume was 150 μL. The cells were incubated at 37° C. in the dark for 6 h.

### 2) Killing Effect of Vγ9Vδ2 T Cells on THP-Treated HCC (Huh7, HepG2) Tumor Cells

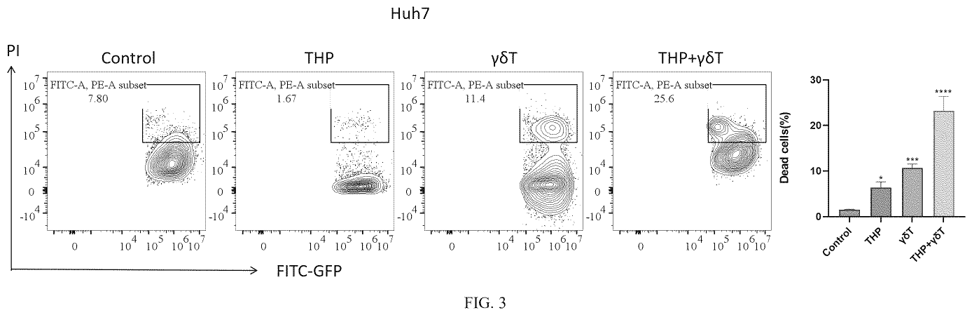

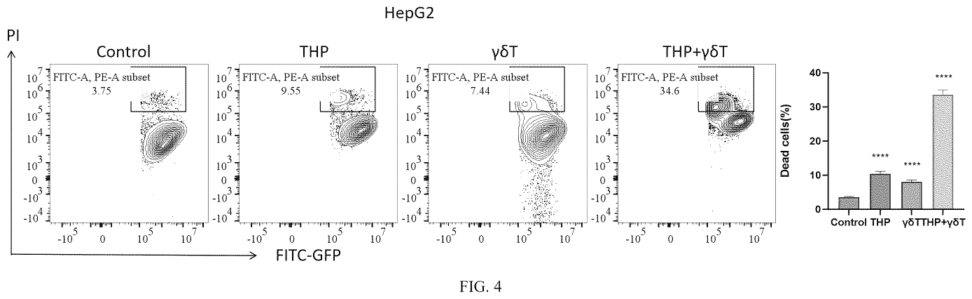

After co-culture, 1 mL PBS was added to resuspend, the cells were centrifuged, and the supernatant was discarded. Each tube was added with 100 μL PBS and 2 μL PI, stained in the dark at 4° C. for 10 min, then resuspended with 400 μL PBS, and analyzed by flow cytometry to detect the killing effect of Vγ9Vδ2 T cells on THP-treated tumor cells (HepG2, Huh7). The results are shown in FIGS. 3 and 4.

FIGS. 3 and 4 show that the killing effect of Vγ9Vδ2 T cells on THP-treated tumor cells (HepG2, Huh7) is significantly increased compared with the blank control, the killing effect of THP on tumor cells, and the killing effect of Vγ9Vδ2 T on tumor cells. This indicates a synergistic effect between the Vγ9Vδ2 T cells and THP.

3) Detection of Effect of THP Combined with Vγ9Vδ2 T Cells on HCC (HepG2 and Huh7) Cell Proliferation by Flow Cytometry

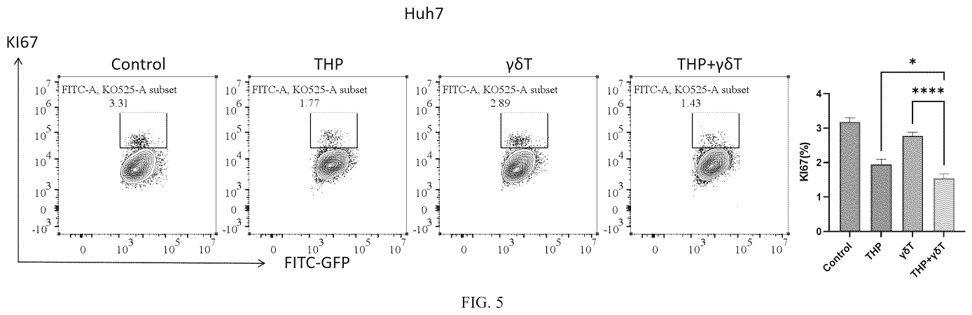

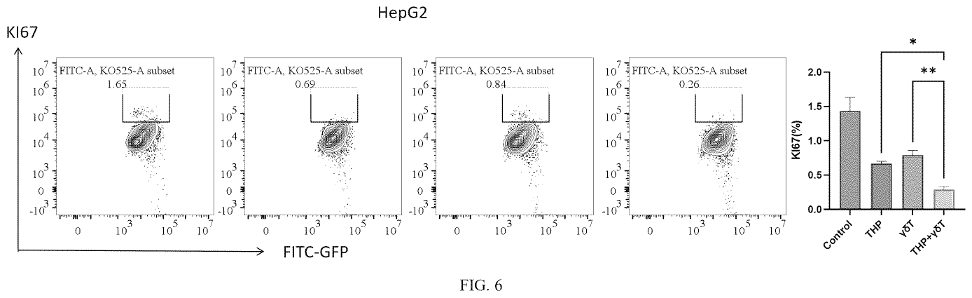

After incubation, the cells in the above blank control group and experimental groups were resuspended with 1 mL PBS and centrifuged; and the supernatant was discarded. Each tube was added with 100 μL PBS and 2.5 μL Ki67, stained in the dark at 4° C. for 10 min, then resuspended with 400 μL PBS, and analyzed by flow cytometry. The results are shown in FIGS. 5 and 6.

The results indicate that THP combined with Vγ9Vδ2 T cells significantly inhibits proliferation of tumor cells.

### Example 3: Effect of THP on MICA and MICB Expression in HCC (Huh7, HepG2) Tumor Cells

1) Detection of Effect of THP on MICA and MICB Expression in HCC (Huh7, HepG2) Tumor Cells by qPCR

### Experimental Grouping

Control group: Tumor cells were plated at 5×10⁵ cells per well in a 12-well plate and incubated at 37° C., 5% CO₂ for 30 h.

THP group: Tumor cells were plated at 5×10⁵ cells per well in a 12-well plate and incubated at 37° C., 5% CO₂ for 18 h. After cell attachment, 2 μM THP was added to treat the cells for 12 h.

RNA was extracted from tumor cells of the Control group and the THP group. Reverse transcription was performed using a reverse transcription kit, followed by cDNA quantification using SYBR Green.

### 2) Detection of Effect of THP on MICA and MICB Expression in HCC (Huh7, HepG2) Tumor Cells by Western Blot

### Experimental Grouping

Control group: Cells were plated at 6×10⁵ cells per well in a 12-well plate and incubated at 37° C., 5% CO₂ for 30 h.

THP group: Cells were plated at 6×10⁵ cells per well in a 12-well plate and incubated at 37° C., 5% CO₂ for 18 h. After cell attachment, 2 μM THP was added to treat the cells for 12 h.

The supernatant was discarded, and each well was washed twice with 1 mL PBS. Then, 200 μL of prepared lysis buffer (RIPA:PMSF:protease inhibitor:phosphatase inhibitor=100:1:1:1) was added per well. Cells were lysed on ice for 5 min and scraped from the well to obtain a cell suspension. The suspension was transferred to an EP tube and lysed on a rotator at 4° C. for 20 min. The lysate was centrifuged at 12,000 rpm for 20 min, and 170 μL of the supernatant was mixed with 85 μL of 3× Loading Buffer (Beyotime), followed by boiling at 100° C. for 10 min. Proteins were separated by SDS-PAGE and transferred to PVDF membranes using a constant current of 300 mA for 2 h. Membranes were blocked in 5% skim milk on a horizontal shaker at room temperature for 2 h. After blocking, membranes were washed with 15 mL TBST for 10 min×3 times. Membranes were incubated with corresponding primary antibodies for MICA/B overnight, washed with TBST for 10 min×3 times, incubated with corresponding secondary antibodies, and washed again for 10 min×3 times. Exposure solution (solution A:solution B=1:1) was evenly added onto the membrane according to the molecular weight of proteins for detection.

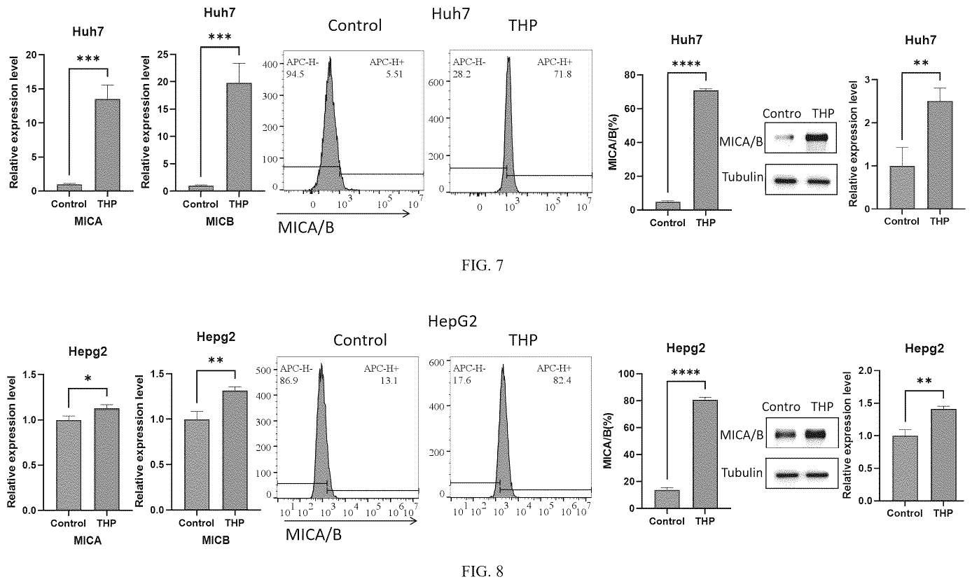

The results of qPCR and Western Blot detecting the effect of THP on MICA and MICB expression in HCC (Huh7, HepG2) tumor cells are shown in FIGS. 7 and 8.

The results indicate that after THP treatment, gene levels of surface NKG2D ligands MICA and MICB in Huh7 and HepG2 hepatocellular carcinoma cell lines are significantly increased.

### Example 4: Effect of THP-Treated HCC (Huh7, HepG2) Tumor Cells on NKG2D Expression on Vγ9Vδ2 T Cells

### Experimental Grouping:

Control group: Tumor cells were plated at 6×10⁵ cells per well in a 6-well plate and incubated at 37° C., 5% CO₂ for 24 h. For each tumor cell type, 6×10⁵ cells were collected and co-cultured with Vγ9Vδ2 T cells at an effector-to-target ratio of 1:2 in round-bottom flow cytometry tubes.

THP group: Tumor cells were plated at 6×10⁵ cells per well in a 6-well plate and incubated at 37° C., 5% CO₂ for 12 h. After cell attachment, 2 μM THP was added to treat the cells for 12 h. For each tumor cell type, 6×10⁵ THP-treated cells were collected and co-cultured with Vγ9Vδ2 T cells at an effector-to-target ratio of 1:2 in round-bottom flow cytometry tubes.

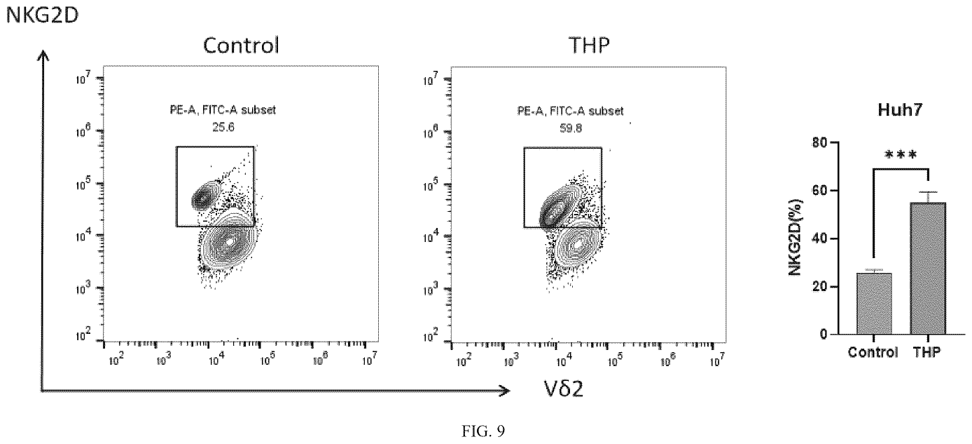

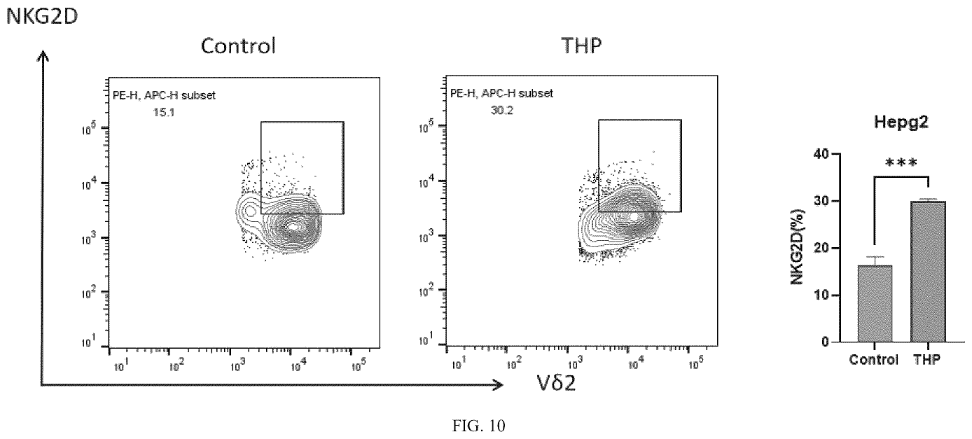

The co-culture volume was 150 μL. The cells were incubated at 37° C. in the dark for 6 h. After co-culture, 1 mL PBS was added to resuspend, the cells were centrifuged, and the supernatant was discarded. Each tube was added with 100 μL PBS, 2 μL PI, and 2 μL NKG2D, stained in the dark at 4° C. for 25 min, then resuspended with 400 μL PBS, and analyzed by flow cytometry. The results are shown in FIGS. 9 and 10.

The results indicate that THP-treated hepatocellular carcinoma cells promote high expression of NKG2D on Vγ9Vδ2 T cells.

### Example 5: Animal Experiments

### 1) Construction of Humanized Mice

C-NKG mice weighing 23±2 g were raised under a clean environment at 25±2° C. Each mouse was intravenously injected via the tail vein with 5×10⁶ PBMCs. Mouse eyeball venous blood was collected once per week. Flow cytometry was used to detect the proportion of CD45. When the CD45 proportion reached over 10% in the second week, the humanized mice were successfully constructed.

### 2) Construction of Hepatocellular Carcinoma Mouse Model

Huh7 cells (5×10⁶ cells/100 μL per mouse) were inoculated subcutaneously into the right hind leg of the successfully constructed humanized mice. Tumor growth was observed for 12-14 days after subcutaneous inoculation. A visible bulge at the injection site indicated successful model construction. The successfully constructed mice were randomly divided into four groups: control group, Vγ9Vδ2 T group, THP group, and THP+Vγ9Vδ2 T group. Each group included 5 mice.

### 3) Mouse Treatment

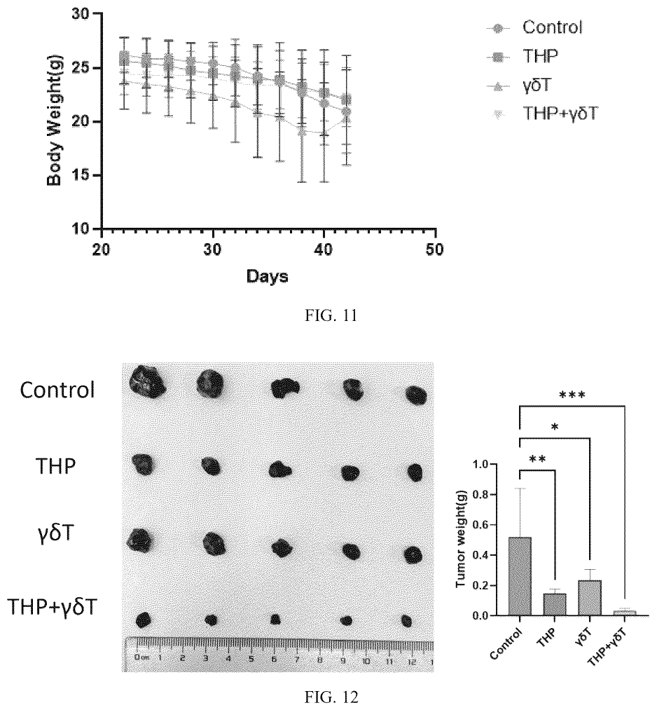

Treatment was performed on the day following successful hepatocellular carcinoma model construction. Control group: Each mouse was injected intratumorally with 100 μL PBS. THP group: Each mouse was injected intratumorally with 2 μg THP+50 μL iodized oil. Vγ9Vδ2 T group: Each mouse was intravenously injected via the tail vein with 5×10⁶ Vγ9Vδ2 T cells. THP+Vγ9Vδ2 T group: Each mouse was injected intratumorally with 2 μg THP+50 μL iodized oil and intravenously injected via the tail vein with 5×10⁶ Vγ9Vδ2 T cells. For the THP+Vγ9Vδ2 T group, after intratumoral injection of 2 μg THP+50 μL iodized oil for 2 days, Vγ9Vδ2 T cells were injected via the tail vein once every 3 days, for a total of 5 injections. Mouse body weight was recorded every other day during the treatment (see Table 1 and FIG. 11). Seven days after the last injection in the THP+Vγ9Vδ2 T group, all mice from all groups were euthanized. Mouse spleens and tumor tissues were collected and weighed; and tumors were arranged from largest to smallest for photography. Tumor tissues were fixed overnight and embedded in paraffin. All animal experiments were performed according to protocols approved by the Ethics Committee of Xinxiang Medical University. Tumor sizes of mice in different groups are shown in FIG. 12.

TABLE 1
Body weight changes of mice during treatment

| | | NC | | | | THP | | | | |
|---|---|---|---|---|---|---|---|---|---|---|
| | | No. | | | | | | | | |
| Days | | 2 | 3 | 4 | 5 | 6 | 7 | 8 | 9 | 10 |
| 22 | 26.1 | 25.99 | 24.57 | 25.43 | 28.92 | 25.62 | 27.12 | 22.43 | 25.02 | 27.99 |
| 24 | 25.05 | 25.85 | 24.3 | 25.2 | 28.88 | 25.2 | 26.98 | 22.01 | 24.91 | 28.07 |
| 26 | 25.88 | 25.7 | 24.02 | 24.95 | 28.65 | 24.75 | 26.77 | 21.67 | 24.88 | 27.78 |
| 28 | 25.72 | 25.56 | 23.7 | 24.63 | 28.43 | 23.98 | 26.58 | 21.02 | 24.64 | 27.54 |
| 30 | 25.54 | 25.43 | 23.29 | 24.2 | 28.56 | 23.58 | 26.24 | 20.8 | 24.51 | 27.3 |
| 32 | 25.69 | 24.88 | 22.33 | 23.18 | 29.08 | 23.58 | 26.02 | 20.04 | 23.96 | 27.69 |
| 34 | 25.05 | 24.2 | 21.85 | 21.81 | 28.31 | 23.29 | 25.25 | 19.11 | 24.35 | 27.82 |
| 36 | 23.82 | 24.22 | 21.62 | 20.52 | 28.16 | 22.35 | 25.15 | 19.04 | 24.61 | 28.32 |
| 38 | 21.99 | 22.91 | 20.9 | 19.61 | 27.85 | 21.62 | 24.21 | 18.44 | 24.37 | 27.6 |
| 40 | 19.83 | 22.17 | 20.22 | 18.15 | 28.09 | 20.85 | 23.33 | 17.16 | 24.21 | 27.87 |
| 42 | 19.78 | 21.53 | 20.25 | 16.2 | 26.92 | 19.68 | 22.28 | 17.02 | 22.91 | 28.17 |
| | γδT | | | | | THP + γδT | | | | |
| | No. | | | | | | | | | |
| Days | 11 | 12 | 13 | 14 | 15 | 16 | 17 | 18 | 19 | 20 |
| 22 | 21.34 | 24.65 | 27.02 | 24.97 | 20.84 | 26.74 | 21.77 | 23.97 | 23.93 | 26.22 |
| 24 | 20.89 | 24.04 | 26.93 | 24.89 | 20.68 | 26.82 | 21.63 | 23.77 | 23.88 | 25.67 |
| 26 | 20.54 | 23.89 | 26.79 | 24.78 | 20.39 | 26.65 | 21.42 | 23.83 | 23.74 | 25.73 |
| 28 | 19.88 | 23.64 | 26.65 | 24.59 | 19.74 | 26.49 | 21.49 | 23.69 | 23.44 | 26.62 |
| 30 | 19.22 | 23.05 | 26.5 | 24.07 | 19.49 | 26.34 | 21.32 | 23.64 | 23.48 | 25.54 |
| 32 | 17.14 | 22.23 | 26.76 | 24.07 | 19.22 | 25.72 | 20.75 | 22.7 | 22.88 | 26.33 |
| 34 | 16.12 | 20.66 | 26.02 | 23.71 | 17.51 | 25.14 | 20.25 | 23.19 | 22.39 | 25.85 |
| 36 | 15.65 | 19.7 | 25.75 | 23.62 | 17.69 | 25.45 | 20.45 | 23.46 | 22.13 | 25.8 |
| 38 | 14.65 | 16.28 | 25.4 | 23.13 | 16.28 | 25.5 | 20.11 | 22.42 | 20.92 | 25.58 |
| 40 | 13.74 | 17.45 | 25.02 | 22.43 | 16.34 | 25.2 | 19.61 | 21.98 | 21.08 | 25.61 |
| 42 | 15.81 | 23.28 | 20.35 | 26.07 | 16.28 | 25.12 | 19.38 | 20.88 | 20.72 | 25.24 |

The results indicate that THP significantly synergizes with Vγ9Vδ2 T cells to inhibit tumor progression.

According to the above examples, THP can increase the killing ability of Vγ9Vδ2 T cells by regulating the NKG2D/MIC A/B pathway, thereby enhancing the antitumor immune response in hepatocellular carcinoma-bearing mice.

The above descriptions are only preferred examples of the present invention. It should be noted that those of ordinary skill in the art can also make several improvements and modifications without departing from the principle of the present invention, and such improvements and modifications shall fall within the protection scope of the present invention.

What is claimed is:

1. A use of Pirarubicin in preparation of a medicament for enhancing anti-hepatocarcinoma efficacy of Vγ9Vδ2 T cells.

2. The use according to claim 1, wherein the Pirarubicin is used in a solution form.

3. The use according to claim 2, wherein a Pirarubicin solution comprises the Pirarubicin and an iodized oil, and a mass-to-volume ratio of the Pirarubicin to the iodized oil is 2 μg:1-100 μL.

4. An anti-hepatocarcinoma medicament, comprising the Pirarubicin in the use according to claim 1 and Vγ9Vδ2 T cells separately packaged.

5. The anti-hepatocarcinoma medicament according to claim 4, wherein the Pirarubicin is used in a solution form.

6. The anti-hepatocarcinoma medicament according to claim 5, wherein a Pirarubicin solution comprises the Pirarubicin and an iodized oil, and a mass-to-volume ratio of the Pirarubicin to the iodized oil is 2 μg:1-100 μL.
